(19) United States
(12) Patent Application Publication
(10) Pub. No.: US 2025/0186248 A1
(43) Pub. Date: Jun. 12, 2025

(54) Cooling Helmet for Preventing Brain Injury

(71) Applicant: Julius Tabe, HOLLISTON, MA (US)

(72) Inventor: Julius Tabe, HOLLISTON, MA (US)

(21) Appl. No.: 18/531,018

(22) Filed: Dec. 6, 2023

(51) Int. Cl. A61F 7/00

(52) U.S. Cl. CPC A61F 7/0085; A61F 2007/0002; A61F 2007/0003; A61F 2007/0007; A61F 2007/0064; A61F 2007/0091

(57) ABSTRACT

A helmet operable to reduce brain temperature, and in turn reduce brain activity, in the event of cardiac arrest is provided. The helmet conveys cold, high-pressure air to the head of the patient in order to reduce the temperature of the brain to, in many instances, a hibernation condition. This greatly limits the consumption of oxygen and glucose by brain tissue, and in turn limits damage caused by lack of blood flow to the brain in a cardiac arrest condition.

### BACKGROUND OF THE INVENTION

The present disclosure relates generally to the field of medical interventions and specifically addresses the critical issue of neurological damage following cardiac arrest. More particularly, the present disclosure relates to a helmet operable to convey cold compressed air to the head of a patient in order to decrease brain temperature and thus brain oxygen consumption in the case of a cardiac arrest event, thus limiting brain damage.

Cardiac arrest is a life-threatening condition where the heart suddenly stops beating, leading to an immediate cessation of blood flow and oxygen supply to vital organs, including the brain. Research has consistently demonstrated that irreversible brain damage begins to occur within a remarkably short period, typically between 4 to 6 minutes after cardiac arrest, due to the lack of oxygen necessary for essential cellular processes.

In the absence of oxygen, brain cells are unable to perform aerobic respiration, a process crucial for converting glucose into the energy required to sustain neuronal activities. The consequential energy deficit leads to widespread cellular dysfunction, ultimately resulting in permanent neurological impairment or even death.

Furthermore, neuronal activities are markedly reduced in low temperatures and may even reduce to full hibernation (zero activity) in freezing temperatures, freezing the brain within seconds of cardiac arrest will instantly hibernate brain cells and eliminate the need for glucose, oxygen, and energy thereby protecting the brain.

Therefore, what is needed is a device that can rapidly reduce the temperature of the brain in the event of a cardiac arrest episode.

### SUMMARY OF THE INVENTION

The subject matter of this application may involve, in some cases, interrelated products, alternative solutions to a particular problem, and/or a plurality of different uses of a single system or article.

In one aspect, a helmet operable to rapidly decrease the temperature of a brain is provided. The helmet comprises an air compressor which is operable to compress inlet air from the atmosphere and discharge the compressed air through an outlet, as well as an air chiller operable to reduce the temperature of air in many cases to sup-freezing or sub-zero temperatures. The chiller has an inlet in communication with the outlet of the air compressor such that after being compressed, the air is then cooled. An outlet of the chiller then connects to the helmet. The helmet is formed generally by a body which defines the helmet's shape as well as a cold-air manifold. The cold air manifold receives the air from the chiller and disperses it through a plurality of vents directed at a head area of a user when the helmet is worn. As such, when worn and activated, the helmet directs a high flow rate of cold air to a wearer. This will draw heat from the head, including the brain, and cause it to rapidly decrease in temperature. This preserves the brain tissue in the event that a patient goes into cardiac arrest and cannot provide blood flow to the brain.

Other aspects of the present disclosure include proper positioning of vents to direct air to areas of the head which more rapidly draw heat from the brain, such as the temples, forehead, rear bottom area of the skull, neck, and the like. Further, air outlets that allow release of the air from the vents may be positioned so as to maximize convection across and over the head and the noted particularly effective areas of the head. Further aspects of the present disclosure are discussed in more detail below.

### BRIEF DESCRIPTION OF DRAWINGS

FIG. 1 provides a perspective view of one embodiment of the present disclosure.

FIG. 2 provides a side view of another embodiment of the present disclosure.

FIG. 3 provides a detail view of an embodiment of the present disclosure.

FIG. 4 provides a detail view of still another embodiment of the present disclosure.

FIG. 5 provides a flow chart of an exemplary embodiment of use of the present disclosure.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns a device which is able to adequately cool down the brain of a user very rapidly—less than the 4-6 minutes in which brain damage occurs after cardiac arrest. The device utilizes a helmet which fits over the head and neck of a patient and which can blow large amounts of very cold air at the head of the patient. This rapidly draws heat out of the brain, chilling it rapidly and reducing its oxygen and nutrient consumption. Optimally, the device will bring the brain to a chilled state within one minute. Further optimally, the device will bring the brain to a chilled state of full or near hibernation within the one minute time frame. Given that neuronal activities are markedly reduced in low temperatures and may even reduce to full hibernation (zero activity) in freezing temperatures, freezing the brain within seconds of cardiac arrest will rapidly hibernate brain cells and eliminate the need for glucose, oxygen, and energy. The helmet is usable in the absence of an adequate Cardiopulmonary Resuscitation team, which usually takes over six minutes to get to patients whose cardiac arrest occur at home or somewhere away from a hospital. The present disclosure provides the ultimate opportunity to preserve the brain's viability until the arrival of trained medical personnel.

The device includes a helmet for covering a patient's head and directing the cold air flow to the skull. The helmet may be a hard shell for protective purposes, or may be soft and flexible, making it more easily fitted on a user. A compressor and chiller serve to pressurize the air and drastically reduce its temperature, in many embodiments decreasing the pressure below freezing (32 degrees Fahrenheit) or below 0 degrees Fahrenheit. In many embodiments, the air is directed all over the skull of a patient when the helmet is applied, however in certain cases, additional air flow may be directed at the temples, forehead, lower rear of the skull, and/or neck which may increase the head transfer from the brain to the air. In certain embodiments, the helmet may have specific outlet vents positioned on the helmet. This may increase convective heat transfer by rapid flow and direction of air. The outlets may be positioned intentionally to draw air out at certain areas, which may increase air flow rates in certain areas, increasing heat transfer.

Chiller devices may be any device able to effectively cool the air. This may involve an air conditioning device, refrigeration device, and the like. Similarly, the compressor may be any device known in the art that may increase the pressure of air, such as a blower.

In still other embodiments, instead of air flow, another fluid may be used such as water, a water-salt solution to lower freezing temperature, a glycol solution and the like. In a particular embodiment, chilled salt water may be directly conveyed to the head of the patient using the helmet having similar structure as described herein. The water may then be collected via tubing at outlets and recycled to a chiller or refrigerator. In some cases, this may provide enhanced heat transfer to the brain. Salt and water are both commonly available at homes and thus may be combined to form an effective low temperature heat transfer medium.

In one embodiment, a sensor or sensors may track operational progress of the cooling process. For example temperature probes may be used. These probes may send a signal to a programmed controller which is able to identify a modeled brain temperature based on temperature sensed and time elapsed of cooled air exposure. The controller may be able to deactivate and reactivate the system periodically to maintain a proper temperature without over-cooling the brain of the patient. In another embodiment, the system may have a programmed controller which operates for a predetermined amount of time, and then pulses on and off after this point once an adequate amount of brain chilling has been achieved. This again may maintain a proper temperature without over-cooling the brain of the patient.

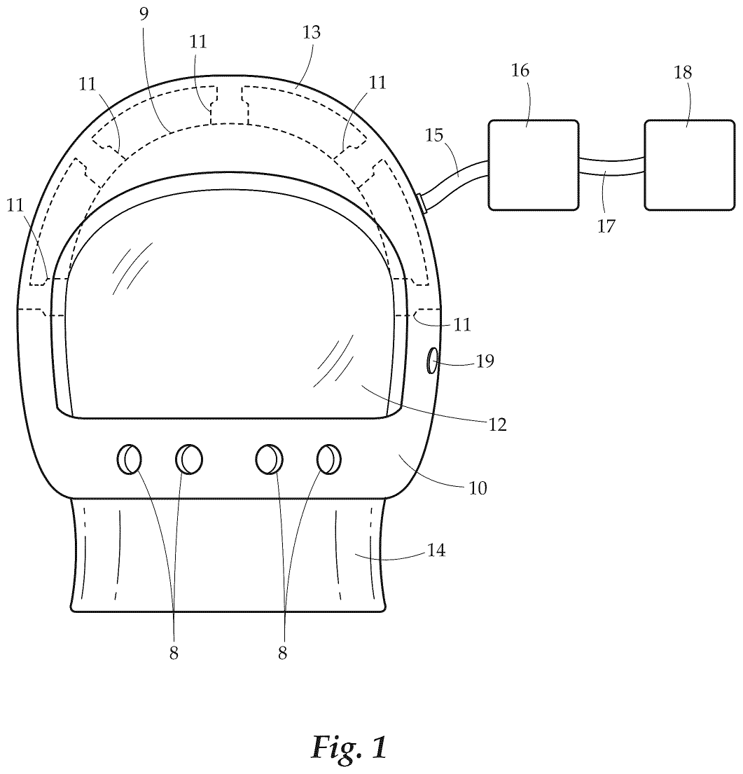

Turning now to FIG. 1 a perspective view of an embodiment of the helmet contemplated herein is shown. The helmet 10 has a compressor 18 and chiller 16 connected thereto, which blows cold air (or other cold fluid) at a head of a patient who is wearing the helmet. The compressor 18 draws air in from the atmosphere and discharges at higher pressure via outlet 17. The outlet 17 enters the chiller 16 which drastically reduces its temperature to below freezing, below zero, and the like. The air after being chilled is discharged via outlet 15 and conveyed to the helmet 10. The helmet 10 defines a cold air manifold 13 which disperses the air through vents 11 on the inner surface 9 of the helmet, directing air towards the head of the user (not shown). A face shield 12 may help seal the helmet to prevent the ingress of warmer ambient air. Similarly, neck cover 14 seals around the neck of the patient to ensure that a cold temperature is maintained around the head within the helmet. The helmet body further defines outlet vents 8 which allow rapid egress of the cold air, and in turn allow for more freshly chilled air to enter. Positioning of the outlet vents 8 aids in convective heat transfer by forming flow paths from inlet vent 11 to outlet 8. Flow path configuration may be directed over high-heat-transfer areas of the head to improve effectiveness of the brain cooling. The system is turned on and off by an on-off switch 19 and is powered by, for example, a battery, a wall plug, and combinations thereof.

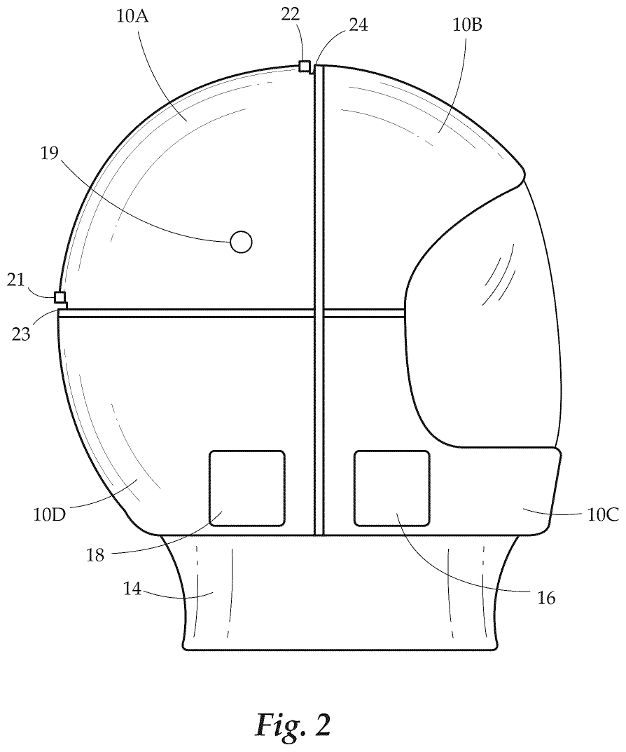

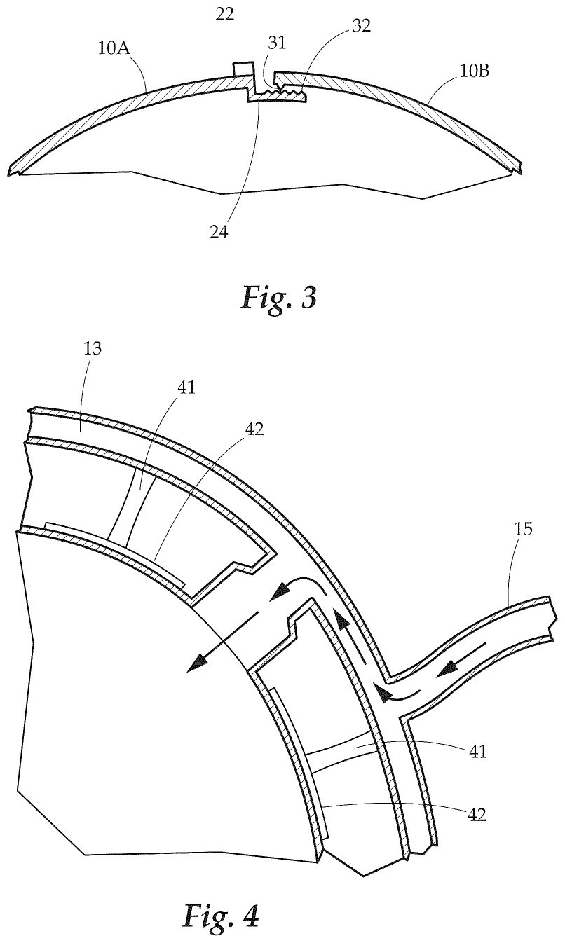

FIG. 2 shows another embodiment of the helmet having a hard shell which is adjustable in two directions. The hard shell of the helmet 10 is formed of four interconnected parts, 10A, 10B, 10C, and 10D. These are connected by a horizontal adjustment flange 24 and a vertical adjustment flange 23 which is connected to one of the parts and fits into or under an adjacent of the parts. An adjuster button 22 depresses a pawl engaged with ratcheting teeth on the horizontal flange 24 and allows adjustment. The arrangement may be reversed, such as shown in FIG. 3 as well without straying from the scope of this disclosure. Similarly, adjuster button 21 can be depressed to release a pawl engaged with ratcheting teeth on the vertical flange 23. Of course, similar embodiments may be used with only one adjustment direction without straying from the scope of this invention. Further, in this embodiment, the compressor 18 and chiller 16 are attached to the helmet body 10.

FIG. 3 provides a detail view of an embodiment of the locking adjustment system of FIG. 2. The button 22 depresses the flange 24 and in turn moves teeth 32 away from the pawl 31, allowing movement of the adjacent helmet parts 10A and 10B.

FIG. 4 provides another embodiment of an adjustment system. In this view, the pressurized inlet air coming from the compressor 18 and chiller 16 is used to inflate an inflatable section 42 to properly size the helmet. Because air or other fluid flow is very important to chilling the brain, the inflatable sections are small and designed to only cover a small part of the surface area of the head of a user when worn. Air is conveyed from the chiller outlet 15 through manifold 13 to the inflatable section(s) 42 via channel or channels 41.

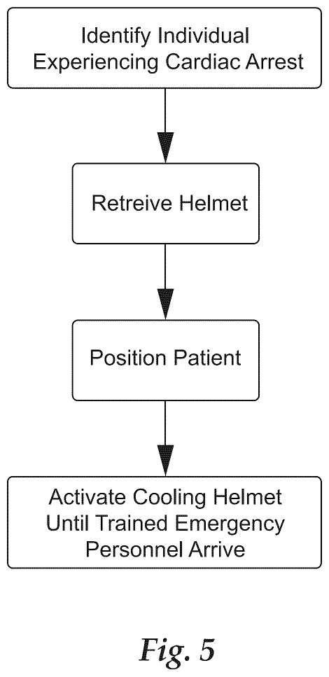

FIG. 5 provides a flow chart of an exemplary embodiment of use of the present disclosure. Initially, an individual experiencing cardiac arrest is identified by a nearby person. An embodiment of the helmet described herein is retrieved and then positioned on the patient. Once fitted and secure, the cooling helmet system is activated and maintained until trained emergency personnel arrive.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A brain-chilling helmet comprising:
an air compressor operable to compress inlet air and discharge the compressed air through an outlet;
an air chiller operable to reduce the temperature of air, the chiller having an inlet in communication with the outlet of the air compressor, and having an outlet;
a helmet, the helmet having a body which defines a cold air manifold and a plurality of vents, each vent in communication with the cold air manifold and positioned at positions on the body corresponding to an area of a head of a patient, the cold air manifold being in communication with the outlet of the air chiller, such that a high pressure chilled air enters the manifold, and is conveyed through the plurality of vents.

2. The brain-chilling helmet of claim 1 further comprising a plurality of outlet vents.

3. The brain-chilling helmet of claim 2 wherein the plurality of outlet vents are positioned to increase convective heat transfer.

4. The brain-chilling helmet of claim 1 further comprising a neck covering connected to a bottom of the helmet body.

5. The brain-chilling helmet of claim 1 wherein the helmet body comprises a hard outer shell.

6. The brain-chilling helmet of claim 1 wherein the helmet body is flexible.

7. The brain-chilling helmet of claim 1 wherein at least two of the plurality of vents are directed at a location corresponding to each temple of the head of the patient.

8. The brain-chilling helmet of claim 1 wherein at least one of the plurality of vents is directed at a location corresponding to a forehead of the head of the patient.

9. The brain-chilling helmet of claim 1 further comprising an air-tight face shield.

10. The brain-chilling helmet of claim 1 wherein the helmet is adjustable in size.

11. The brain-chilling helmet of claim 10 wherein an air pressure from the compressor in the cold air manifold is operable to inflate a portion of the body to adjust the size of the helmet.

12. The brain-chilling helmet of claim 11 wherein the portion of the body to be inflated is a flexible bladder and the flexible bladder in communication with the cold air manifold via an inflation channel.

13. The brain-chilling helmet of claim 10 wherein the body comprises a plurality of adjustable sections that are movable relative to each other and lockable in a plurality of different positions.

14. The brain-chilling helmet of claim 10 wherein the body is adjustable by a ratchet and pawl assembly.

15. The brain-chilling helmet of claim 1 wherein the chiller and compressor are mounted to the body of the helmet.

16. The brain-chilling helmet of claim 1 wherein the chiller and compressor are separate from the body of the helmet.

17. The brain-chilling helmet of claim 1 wherein the chiller is operable to reduce a temperature of the outlet air to below freezing.

18. The brain-chilling helmet of claim 1 wherein the chiller is operable to reduce a temperature of the outlet air to below zero degrees Fahrenheit.

19. The brain-chilling helmet of claim 1 further comprising a battery power source in electrical communication with the chiller and compressor.

20. The brain-chilling helmet of claim 1 further comprising a power plug power source in electrical communication with the chiller and compressor.
